(19) United States
(12) Patent Application Publication
(10) Pub. No.: US 2017/0119794 A1
(43) Pub. Date: May 4, 2017

(54) TREATMENTS OF ACCUMULATED FAT WITH DEOXYCHOLIC ACID AND SALTS THEREOF

(71) Applicant: Kythera Biopharmaceuticals, Inc., Calabasas, CA (US)

(72) Inventors: Frederick Beddingfield, Calabasas, CA (US); Elisabeth Sandoval, Calabasas, CA (US); Nancy Jorgesen, Calabasas, CA (US); Serge Lichtsteiner, Calabasas, CA (US); Paul Lizzul, Calabasas, CA (US); Todd Gross, Calabasas, CA (US); Christine Somogyi, Calabasas, CA (US)

(73) Assignee: Kythera Biopharmaceuticals, Inc., Calabasas, CA (US)

(21) Appl. No.: 15/344,489

(22) Filed: Nov. 4, 2016

Related U.S. Application Data
(60) Provisional application No. 62/251,014, filed on Nov. 4, 2015.

(51) Int. Cl. A61K 31/575; A61K 47/10

(52) U.S. Cl. CPC A61K 31/575; A61K 47/10

(57) ABSTRACT

Provided herein are methods of treating a variety of disorders related to fat accumulation in humans with pharmaceutical formulations containing deoxycholic acid.

### CROSS-REFERENCE TO RELATED APPLICATIONS

This application claims priority under 35 U.S.C. §119(e) to U.S. Provisional Application Ser. No. 62/251,014, filed Nov. 4, 2015, the entirety of which is incorporated herein by reference.

### FIELD OF THE INVENTION

This invention relates to methods of treating a variety of disorders related to fat accumulation in humans with aqueous pharmaceutical formulations containing deoxycholic acid (“DCA”), preferably low or very low concentrations of a salt of DCA. In a preferred embodiment, the pharmaceutical composition is buffered to maintain a physiologically acceptable pH such that the composition is suitable for injection, such as to the site of fat accumulation.

### BACKGROUND

Published literature reports that the aqueous solutions of DCA has fat removing properties when injected into fatty deposits in vivo (See, WO 2005/117900 and WO 2005/112942, US2005/0261258; US2005/0267080; US2006/127468; and US2006/0154906, each of which is incorporated herein in its entirety by reference). DCA injected into fat tissue degrades fat cells via a cytolytic mechanism to provide the desired aesthetic results.

Notwithstanding the benefits of aqueous formulations of DCA, it has been found that at low concentrations of DCA (i.e., less than or about 2% w/v) in aqueous solutions which optionally contain benzyl alcohol, forms a precipitate after storage over a period of time. Surprisingly, it has been found that the lower the concentration DCA, the higher is the rate of precipitation notwithstanding any significant change in the pH of the solution. This precipitation at very low concentrations is a problem for commercialization as a precipitate is counter-indicated for subcutaneous injections of DCA.

In each treatment regimen, the current clinical trials of aqueous formulations of DCA employ multiple injections of small amounts of the aqueous formulation into different sites defining the fat deposit to be treated.

As is apparent, aqueous formulations of DCA used in such treatments overlap with the problems arising from precipitation of the DCA. That is to say that an initially clear aqueous solution of DCA when stored for a period of time, will form a precipitate at commercially relevant concentrations of DCA notwithstanding the fact that the pH of these solutions are between about 7.50 and about 8.0 which are substantially above the pKa of deoxycholic acid.

There is a need for treating a variety of disorders related to fat accumulation in humans with DCA, preferably low concentrations of a salt of DCA, more preferably with a low concentration, aqueous solution of deoxycholic acid or a salt thereof, stabilized against precipitation during a shelf life of at least 2 months.

### SUMMARY OF THE INVENTION

In one aspect, provided herein is a method of treating accumulated fat in a patient in need thereof, comprising administering into the accumulated fat a therapeutically effective amount of DCA or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea.

In another aspect, provided herein is a method of treating accumulated fat in a patient in need thereof, comprising administering into the accumulated fat a therapeutically effective amount of DCA or a pharmaceutically acceptable salt thereof and at least one pharmaceutically acceptable excipient, wherein the accumulated fat results from or causes one or more of bra fat, back of arm fat, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including periumbilical fat), excess fat on the buttocks, mons pubis fat, excess fat around the ankles, lipoma, lipodystrophy (such as Dunning-type lipodystrophy), lipomatosis such as familial multiple lipomatosis, post-liposuction fat deposits, and obstructive sleep apnea.

In another aspect, provided herein is a method of reducing accumulated fat in a patient in need thereof, comprising administering into the accumulated fat a therapeutically effective amount of DCA or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the back or buttocks, mons pubis fat, excess fat around the ankles, pseudogynocomastia fat, lipoma, lipodystrophy (such as Dunning-type lipodystrophy), lipomatosis such as familial multiple lipomatosis, post-liposuction fat deposits, and obstructive sleep apnea.

In another aspect, provided herein is a method of reducing accumulated fat in a patient in need thereof, comprising administering into the accumulated fat a therapeutically effective amount of DCA or a pharmaceutically acceptable salt thereof and at least one pharmaceutically acceptable excipient, wherein the accumulated fat results from or causes one or more of bra fat, back of arm fat, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including periumbilical fat), excess fat on the back or buttocks, mons pubis fat, excess fat around the ankles, lipoma, lipodystrophy (such as Dunning-type lipodystrophy), lipomatosis such as familial multiple lipomatosis, post-liposuction fat deposits, and obstructive sleep apnea.

In some embodiments, the lipodystrophy results from the patient further suffering from human immunodeficiency virus (HIV). In some embodiments, the lipodystrophy is Madelung's disease.

In some embodiments, the DCA is administered by injection. In some embodiments, the DCA is administered by subcutaneous injection. In some embodiments, the DCA is administered by a plurality of injections. In some embodiments, the DCA is administered by a plurality of subcutaneous injections.

In various embodiments, the following formulations are useful according to the invention.

In one embodiment, the DCA is administered as an aqueous formulation.

In one embodiment, the aqueous formulations consists essentially of a salt of deoxycholic acid at a concentration of from about 0.4% w/v to less than about 2% w/v and optionally a preservative effective amount of benzyl alcohol which formulations are stabilized against precipitation by adjusting the pH of the initially formed clear solution to a pH of from about 8.1 to about 8.5. In another embodiment, the aqueous formulation consists essentially of a salt of deoxycholic acid at a concentration of from about 0.5% w/v to about 1% w/v and optionally a preservative effective amount of benzyl alcohol which formulations are stabilized against precipitation by adjusting the pH of the initially formed clear solution to a pH of from about 8.1 to about 8.5.

In another embodiment, the aqueous formulation consisting essentially of:

a sterile aqueous solution buffered to a pH of about 8.3;

about 0.5% w/v or about 1% w/v of a salt of deoxycholic acid;

optionally a preservative effective amount of benzyl alcohol; and

about 1% w/v of sodium chloride,

wherein the composition is stable against precipitation.

Also disclosed herein is a method to lyse a fat cell in the accumulated fat of the patient, comprising administering to said cell a composition according to this invention.

### BRIEF DESCRIPTION OF THE DRAWING

FIG. 1 illustrates various forms or effects of accumulated fat in a patient.

### DETAILED DESCRIPTION

As used herein, certain terms have the following defined meanings.

All numerical designations, e.g., pH, temperature, time, concentration, and molecular weight, including ranges, are approximations which are varied (+) or (−) by increments of 0.1. It is to be understood, although not always explicitly stated that all numerical designations are preceded by the term “about”. If there are uses of the term which are not clear to persons of ordinary skill in the art, given the context in which it is used, “about” will mean up to plus or minus 10% of the particular term. The term “about” also includes the exact value “X” in addition to minor increments of “X” such as “X+0.1” or “X−0.1.” It also is to be understood, although not always explicitly stated, that the reagents described herein are merely exemplary and that equivalents of such are known in the art.

As used herein, the term “comprising” is intended to mean that the compositions and methods include the recited elements, but do not exclude others.

“Consisting essentially of” when used to define compositions and methods, shall mean excluding any active ingredients. An “active ingredient” is a substance intended to furnish pharmacological activity or other direct effect in the diagnosis, cure, mitigation, treatment, or prevention of disease or to affect the structure or any function of the human body. Thus, for example, a composition consisting essentially of the elements as defined herein would not exclude trace contaminants from the isolation and purification method and pharmaceutically acceptable carriers, such as phosphate buffered saline, preservatives, and the like but would exclude enzymes such as phosphatases, and proteins. Non-limiting examples of such proteins are heparin, albumin, and the like

“Consisting of” shall mean excluding more than trace elements of other ingredients and substantial method steps for administering the compositions of this invention. Embodiments defined by each of these transition terms are within the scope of this invention.

As used herein, the term “salt of deoxycholic acid” or “a salt thereof” refers to pharmaceutically acceptable salts of (4R)-4-((3R,5R,10 S,12S,13R,17R)-3,12-dihydroxy-10,13-dimethylhexadecahydro-1H-cyclopenta[a]phenanthren-17-yl)pentanoate having an alkali metal or an ammonium ion as the cation. Preferred are alkali metal salts, with sodium salts being more preferred.

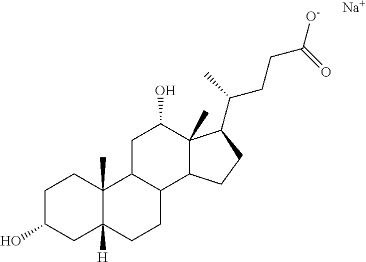

Sodium deoxycholate or sodium (4R)-4-((3R,5R,10S,12S,13R,17R)-3,12-dihydroxy-10,13-dimethylhexadecahydro-1H-cyclopenta[a]phenanthren-17-yl)pentanoate can be prepared according to the methods disclosed in PCT/US2010/061150 titled “Methods for the Purification of Deoxycholic Acid,” filed on Dec. 17, 2010.

As used herein, the term “aqueous pharmaceutical formulation” refers to a pharmaceutically acceptable composition of a deoxycholic acid or a salt thereof in water for administration to a patient preferably via subcutaneous injection from a syringe.

As used herein, the term “buffer” refers to an aqueous solution comprising a mixture of a weak acid and its conjugate base or a weak base and its conjugate acid. A buffer has the property that the pH of the solution changes very little when a small amount of acid or base is added to it. Buffer solutions are used as a means of keeping pH at a nearly constant value in a wide variety of chemical applications. Examples of suitable buffers include phosphate buffers and those known in the literature (see, for example, Troy, D. B., ed. (2005) Remington: The Science and Practice of Pharmacy, 21^{st} ed., Lippincott Williams & Wilkins).

As used herein, the term “base” refers to various typically water-soluble compounds, molecules or ions that in solution have a pH greater than 7. Such compounds, molecules or ions are able to take up a proton from an acid or are able to give up an unshared pair of electrons to an acid. Examples of suitable bases include metal carbonates and bicarbonates, for example sodium carbonate, calcium carbonate, magnesium carbonate, zinc carbonate, sodium bicarbonate and the like; and metal hydroxides, for example sodium hydroxide, potassium hydroxide, and the like, such as those known in the literature (see, for example, Troy, D. B., ed. (2005) Remington: The Science and Practice of Pharmacy, 21^{st} ed., Lippincott Williams & Wilkins).

As used herein, the term “metal carbonates” refers to the metal salt of CO₃²⁻. For example, sodium carbonate, calcium carbonate, magnesium carbonate, zinc carbonate, and the like.

As used herein, the term “metal bicarbonates” refers to the metal salt of HCO₃⁻. For example, sodium bicarbonate, and the like.

As used herein, the term “metal hydroxides” refers to the metal salt of ⁻OH. For example, sodium hydroxide, potassium hydroxide, and the like.

As used herein, the terms “sterile water” or “water for injection” refer to a sterile, nonpyrogenic preparation of water for injection which contains no bacteriostat, antimicrobial agent or added buffer. In general, the osmolar concentration of additives totals at least 112 mOsm/liter (two-fifths of the normal osmolarity of the extracellular fluid −280 mOsm/liter).

As used herein, the term “benzyl alcohol” refers to the compound

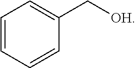

As used herein, the term “precipitation” refers to the formation of a solid in a solution and is readily differentiated from gel formation.

As used herein, the term “solution” refers to a substantially homogeneous mixture comprising two or more substances dissolved in a solvent.

As used herein, the terms “substantially inhibit precipitation” and “inhibits precipitation” means to inhibit most or all visible precipitation so as to maintain homogeneity for a period of time ranging from at least 1 month to at least 1 year.

As used herein, the term “relative standard deviation for homogeneity” or “H_{E}” refers to the value obtained by dividing the standard deviation of the homogeneity by the absolute value of the mean. An H_{E} less than 10 indicates very good homogeneity.

As used herein, the term “therapeutically effective amount” or “therapeutic amount” refers to an amount of a drug or an agent that when administered to a patient suffering from a condition, will have the intended therapeutic effect, e.g., alleviation, amelioration, palliation or elimination of one or more manifestations of the condition in the patient. The therapeutically effective amount will vary depending upon the subject and the condition being treated, the weight and age of the subject, the severity of the condition, the particular composition or excipient chosen, the dosing regimen to be followed, timing of administration, the manner of administration and the like, all of which can be determined readily by one of ordinary skill in the art. The full therapeutic effect does not necessarily occur by administration of one dose, and may occur only after administration of a series of doses. Thus, a therapeutically effective amount may be administered in one or more administrations. For example, and without limitation, a therapeutically effective amount of an agent, in the context of treating accumulated fat, refers to an amount of the agent that reduce or eliminate one or more manifestations of accumulated fat in the patient.

As used herein, the term “treatment”, “treating”, and “treat” are defined as acting upon a disease, disorder, or condition with an agent to reduce or ameliorate the harmful or any other undesired effects of the disease, disorder, or condition and/or its symptoms and produce beneficial or desired clinical results. Treatment, as used herein, covers the treatment of a human patient, and includes: (a) impeding the development of the condition, and/or (b) relieving the condition, i.e., causing regression of the condition and/or relieving one or more symptoms of the condition. For purposes of this invention, beneficial or desired clinical results include, but are not limited to, reducing or eliminating accumulated fat.

As used herein, the term “administration” refers to introducing an agent into a patient. A therapeutic amount can be administered, which can be determined by the treating physician or the like. The related terms and phrases “administering” and “administration of”, when used in connection with a compound or pharmaceutical composition (and grammatical equivalents) refer both to direct administration, which may be administration to a patient by a medical professional or by self-administration by the patient, and/or to indirect administration, which may be the act of prescribing a drug. For example, a physician who instructs a patient to self-administer a drug and/or provides a patient with a prescription for a drug is administering the drug to the patient. In any event, administration entails delivery to the patient of the drug.

As used herein, the term “patient” refers to a human who desires to reduce his or her accumulated fat.

As used herein, the term “lipoma” refers to a benign tumor of fatty tissue.

As used herein, the term “lipomatosis” is an autosomal dominant condition in which multiple lipomas are present on the body.

As used herein, the term “obstructive sleep apnea” refers to sleep apnea that occurs when there are repeated episodes of complete or partial blockage of the upper airway during sleep. Sleep apnea may be more common among people with thick or large necks. The condition is also more common among people who have smaller airways in their noses, throats, or mouths. The small airway could be related to the actual size and shape of the airway, or to obstructions or other medical conditions that are causing obstructions.

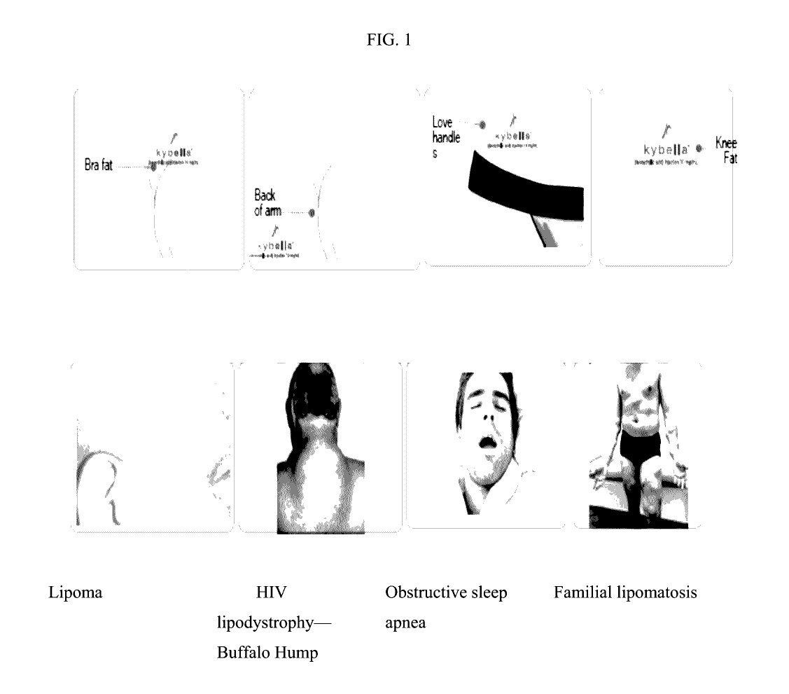

As used herein, the term “bra fat” refers to fat getting squeezed by a bra up and out of the bra causing visible fat rolls or fat bulges. Bra fat may also refer to excess axillary fat, such as lateral periaxillary fat, pre axillary fat, and/or post axillary fat. See, e.g., and without limitation, FIG. 1. In some embodiments, bra fat includes one or more of excess axillary fat, lateral periaxillary fat, pre axillary fat, post axillary fat, anterior periaxillary fat, posterior periaxillary fat, and fat on the upper back.

As used herein, the term “love handle” refers to deposits of excess fat at the sides of a person's waistline. Love handle can also refer to fat on the anterolateral flank.

For the purposes of the present technology, a non-surgical method of fat removal does not include liposuction, lipoplasty or suction lipectomy. As used herein, “non-surgical” refers to medical procedures that do not require an incision. Injections are non-limiting examples of non-surgical procedures. In some embodiments, a method described herein excludes surgical intervention.

### Formulations

In the discussion below, sodium deoxycholate is recited for illustrative purposes only and it is understood that other pharmaceutically acceptable salts of deoxycholic acid can be used interchangeably with the sodium salt.

Current clinical methods for the administration of a sodium deoxycholate to a patient to dissolve fat include the administration via subcutaneous injections of a low concentration (i.e., <2% w/v) of an aqueous solution of the salt of deoxycholic acid where the amount of the salt of deoxycholic acid is sufficient to lyse fat cells (about 0.4% w/v and higher). At such concentrations, it has been shown that the low concentration is beneficial for the effective and safe removal of fat deposits in the body. However, it has been observed that a precipitate forms at such relatively low concentrations of sodium deoxycholate in aqueous media. This precipitation results in a limited shelf life of aqueous solutions of sodium deoxycholate, even at cold temperatures (3-5° C.). In one embodiment, the sodium salt can be replaced by another alkali metal salt.

This instability of aqueous solutions of sodium deoxycholate can be circumvented by the preparation of an aqueous solution of sodium deoxycholate at a concentration of about 5% to about 16% w/v, and having the practitioner dilute the pharmaceutical composition of the sodium deoxycholate solution just prior to use. Whereas this dilution method is effective to allow for both storage stability and effective patient dosing, it is not ideal as a method for routine use especially if a sterile injectable solution of no more than about 2 mL is required. Moreover, current clinical plans include up to 50 injections per treatment session.

It has been found that aqueous formulations of sodium deoxycholate at concentrations ranging from about 0.4% w/v to less than about 2% w/v can be stabilized by adjusting the pH of the solution. In some embodiments, this invention utilizes an aqueous formulation consisting essentially of a salt of deoxycholic acid at a concentration ranging from about 0.4% w/v to less than about 2% w/v and optionally a pharmaceutically acceptable excipient such as a preservative effective amount of benzyl alcohol and/or a pH adjusting buffer, wherein said formulation is maintained at a pH of about 8.1 to about 8.5. Use of other DCA and DCA salt formulations, such as those with higher and lower DCA concentrations and/or with higher or lower pH are also contemplated according to this invention, to the extent, the DCA or DCA salt is soluble or substantially soluble in the formulation and/or the pH of the formulation is suitable for administration into accumulated fat of a human patient. Accordingly, in some embodiments, a concentration of a salt of deoxycholic acid ranging from about 0.1% w/v to about 10% w/v, such as about 0.2% w/v to about 5% w/v, or 0.3% w/v to about 3% w/v is contemplated for use according to this invention. In some embodiments, a pH of about 6.5-about 8.5 for the DCA formulation is contemplated for use according to this invention. In some embodiments, the pH of the DCA formulation is from about 8.0 to about 8.5. In some embodiments, the pH of the DCA formulation is from about 8.0 to about 8.4. In some embodiments, the pH of the DCA formulation is from about 8.1 to about 8.5. In some embodiments, the pH of the DCA formulation is from about 8.1 to about 8.4.

In another embodiment, the aqueous formulation is lyophilized to provide for a stable composition which is ready to be reconstituted by addition of the appropriate amount of water. In this embodiment, this invention comprises lyophilized compositions as described above which optionally further contain a lyophilization aid.

In one embodiment, the aqueous formulation contains about 0.5% w/v of a salt of deoxycholic acid. In another embodiment, the aqueous formulation contains about 1% w/v of a salt of deoxycholic acid.

In a further embodiment, the water employed in the aqueous formulation is sterile water. In still a further embodiment, the preservative effective amount of benzyl alcohol is about 0.9% w/v benzyl alcohol and the pH of the formulation is about 8.3. In one embodiment, said salt is an alkali metal salt. In another embodiment, said salt is a sodium salt.

In one embodiment, the pharmaceutical formulations disclosed herein are suitable for injection into a human. The method of injection can be any type of injection, such as subcutaneous injection, as well as other forms of injection.

In one preferred aspect of this invention, the precipitation of the salt of deoxycholic acid in the aqueous formulation is inhibited for a period of at least about six months. In another aspect, the precipitation is inhibited for a period of at least about one year. In yet another aspect, the precipitation is inhibited for a period of at least about two years.

It is contemplated that when stored at various temperatures, for example at ambient or cold temperatures, the formulation can have an increased shelf life. In certain embodiments, the composition is stored at a temperature of from about 17° C. to about 27° C. In some embodiments, the temperature of the formulation is increased to a temperature of about 25° C. to about 37° C. In other embodiments, the formulation is stored at a temperature of from about 2° C. to about 8° C.

In certain embodiments, the pH of the formulation ranges from about 8.1 to about 8.5. In one embodiment, the pH of the composition is about 8.1, or alternatively, about 8.2, or alternatively, about 8.3, or alternatively, about 8.4, or alternatively, about 8.5. In a preferred embodiment, the pH of the formulation is about 8.3.

In one embodiment, the pH is established by the use of a base. It is contemplated that any base can be used to increase the pH of the composition provided that it does not react with the sodium deoxycholate and will not cause harm to the patient. In some embodiments, the base is selected from the group consisting of metal carbonates, metal bicarbonates, metal hydroxides, or a mixture thereof. Examples of bases include, but are not limited to, a base selected from the group consisting of sodium carbonate, calcium carbonate, magnesium carbonate, zinc carbonate, sodium bicarbonate, sodium hydroxide and potassium hydroxide or a mixture thereof. In one embodiment, the base is sodium hydroxide.

In certain cases, the pH of the composition may be maintained at the desired pH during storage with the use of a buffer. Various buffers are known in the art and it is contemplated that any buffer having buffering capacity at the desired pH can be used in the formulations disclosed herein. In one embodiment, the buffer is a phosphate buffer. The amount of phosphate in the composition can be determined to provide a desired pH and salt concentration. In one embodiment, the composition comprises about 10 mM phosphate buffer. In a preferred embodiment, the composition comprises about 10 mM dibasic sodium phosphate buffer.

In some embodiments, the composition comprises at least one excipient to aid in achieving a composition with desired properties, such as increased solubility, preservability or to provide an isotonic solution. Such excipients (pharmaceutically acceptable and/or cosmetically acceptable) are known in the art. Pharmaceutically acceptable and/or cosmetically acceptable excipients include any and all solvents, diluents or other liquid vehicles, dispersion or suspension aids, surface active agents, isotonic agents, thickening or emulsifying agents, preservatives, solid binders, lubricants and the like, as suited to the particular dosage form desired. Pharmaceutically acceptable and/or cosmetically acceptable excipients include, but are not limited to, benzyl alcohol, sodium chloride, buffer, and water. In one embodiment, the composition comprises about 1% w/v sodium chloride. In another embodiment, the composition comprises about 0.9% w/v benzyl alcohol. In some embodiments, the composition comprises about 0.9% w/v benzyl alcohol and about 1% w/v sodium chloride.

In some embodiments, the pH of the composition is established by use of a base and optionally maintained by use of a buffer.

In a preferred embodiment, this invention utilizes a stabilized composition comprising:

a phosphate buffer of a pH of about 8.3;

about 0.5% w/v or about 1% w/v of sodium deoxycholate;

a preservative effective amount of benzyl alcohol; and

about 1% w/v of sodium chloride,

wherein the composition is stabilized against precipitation.

In a further embodiment, the phosphate buffer is 10 mM dibasic sodium phosphate buffer.

In one embodiment, the preservative effective amount of benzyl alcohol is about 0.9% w/v.

The formulations utilized herein comprise from about 0.4% w/v to less than about 2% w/v of a salt of deoxycholic acid in water maintained at a pH sufficient to substantially inhibit precipitation of the salt of deoxycholic acid. The amount of precipitation or homogeneity of the composition can be measured using various methods. For example, it can be measured quantitatively using light scattering by illuminating the composition with a spectrophotometer. Or alternatively, the homogeneity can be measured qualitatively by observing the visual clarity of the solution with the eye. In some embodiments, the composition has a relative standard deviation for homogeneity of less than about 5%. Alternatively, the composition has a relative standard deviation for homogeneity of less than about 4%, or alternatively, about 3%, or alternatively, about 2%, or alternatively, about 1%.

In another embodiment, this invention utilizes a composition consisting essentially of:

a sterile aqueous solution buffered to a pH of about 8.3;

about 0.5% w/v or 1% w/v of sodium deoxycholate;

about 0.9% w/v benzyl alcohol; and

about 1% w/v of sodium chloride,

wherein the composition is stable against precipitation.

In another embodiment, this invention is directed to a composition consisting of:

an aqueous solution buffered to a pH of about 8.3;

about 0.5% w/v or about 1% w/v of sodium deoxycholate;

about 0.9% w/v benzyl alcohol; and

about 1% w/v of sodium chloride,

wherein the composition is stable against precipitation.

In another embodiment, this invention utilizes a composition consisting essentially of:

a sterile aqueous solution buffered to a pH of about 8.3;

about 0.5% w/v or 1% w/v of sodium deoxycholate;

about 0.9% w/v benzyl alcohol; and

about 0.44% w/v of sodium chloride,

wherein the composition is stable against precipitation.

In another embodiment, this invention is directed to a composition consisting of:

an aqueous solution buffered to a pH of about 8.3;

about 0.5% w/v or about 1% w/v of sodium deoxycholate;

about 0.9% w/v benzyl alcohol; and

about 0.44% w/v of sodium chloride,

wherein the composition is stable against precipitation.

In another embodiment, this invention utilizes a composition consisting essentially of:

a sterile aqueous solution buffered to a pH of about 8.3;

about 1% w/v of sodium deoxycholate;

about 0.9% w/v benzyl alcohol; and

about 0.44% w/v of sodium chloride,

wherein the composition is stable against precipitation.

In another embodiment, this invention is directed to a composition consisting of:

an aqueous solution buffered to a pH of about 8.3;

about 1% w/v of sodium deoxycholate;

about 0.9% w/v benzyl alcohol; and

about 0.44% w/v of sodium chloride,

wherein the composition is stable against precipitation.

In another embodiment, this invention utilizes a composition consisting essentially of:

a sterile aqueous solution buffered to a pH of about 8.3;

about 0.5% w/v or 1% w/v of sodium deoxycholate; and

about 0.75% w/v of sodium chloride,

wherein the composition is stable against precipitation.

In another embodiment, this invention is directed to a composition consisting of:

an aqueous solution buffered to a pH of about 8.3;

about 0.5% w/v or about 1% w/v of sodium deoxycholate; and

about 0.75% w/v of sodium chloride,

wherein the composition is stable against precipitation.

In another embodiment, this invention utilizes a composition consisting essentially of:

a sterile aqueous solution buffered to a pH of about 8.3;

about 1% w/v of sodium deoxycholate; and

about 0.75% w/v of sodium chloride,

wherein the composition is stable against precipitation.

In another embodiment, this invention is directed to a composition consisting of:

an aqueous solution buffered to a pH of about 8.3;

about 1% w/v of sodium deoxycholate; and

about 0.75% w/v of sodium chloride,

wherein the composition is stable against precipitation.

In some embodiments, the solutions herein do not include lipids, phospholipids, or phosphatidylcholine. In some embodiments, the solutions herein include up to 5% w/w, w/v, or v/v lipids, specifically phospholipids, or more specifically phosphatidylcholine. Preferably, the amount of lipids used is less than that of sodium deoxycholate or another salt of deoxycholic acid. In some embodiments, the solution is devoid of phosphatidylcholine.

In some embodiments, the aqueous pharmaceutical composition utilized in the invention can further comprise a second therapeutic agent selected from the group consisting of: anti-microbial agents, vasoconstrictors, anti-thrombotic agents, anti-coagulation agents, suds-depressants, anti-inflammatory agents, analgesics, dispersion agents, anti-dispersion agents, penetration enhancers, steroids, tranquilizers, muscle relaxants, and anti-diarrhea agents. In some embodiments, a solution is in a container that contains up to 500 mL of solution. Such container can be a syringe or syringe-loadable container.

In some embodiments, the formulations further comprise a molecule known to cause fat to die by an orthogonal mechanism. Such molecules include neuropeptide Y (NPY) antagonists including, but not limited to, NPY receptor antagonists, such as BIBP-3226 (Amgen), BIBO-3304 (Boehringer Ingleheim), BMS-192548 and AR-H040922 (Bristol-Myers Squibb), LY-357897 (Eli Lilly), 1229U91 and GW4380145 (GlaxoSmithKline), JNJ-5207787 (Johnson & Johnson), Lu-AA-44608 (Lundbeck), MK-0557 (Merck NPY), NGD-95-1 (Neurogen), NLX-E201 (Neurologix), CGP-71683 (Novartis), PD-160170 (Pfizer), SR-120819A, BIIE0246, and S.A. 0204 (Sanofi Aventis), S-2367 (Shiongli), dihydropyridine and dihydropyridine derivatives that are NPY receptor antagonists, bicyclic compounds that are NPY receptor antagonists, carbazole NPY receptor antagonists, and tricyclic compounds that are NPY receptor antagonists (See, e.g., WO 2006/133160 and U.S. Pat. No. 6,313,128). Also contemplated are fat selective pro-apoptotic peptides such as the CKGGRAKDC peptide that homes to white fat vasculature (See, Kolonin M. G. et al., Nat. Med., 2004, 10(6): 625-32).

Another aspect of the invention utilizes mixing adipo-ablative bile acids, such as, deoxycholic acid (DCA) with agents that kill fat cells. In one aspect, this invention contemplates a means to enhance the aesthetic effects of deoxycholate injections by mixing into the deoxycholate injectate a molecule that causes fat to die by an orthogonal mechanism. Examples of such candidate molecules include, but are not limited to, neuropeptide Y (NPY) antagonists and fat selective pro-apoptotic peptides. Since fat cell killing may be required to mediate the desired effects, the effects of an agent with fat killing ability can be enhanced via the addition of a molecule with potent fat cell killing effects. Additionally, molecules that require access to the vasculature to kill (such as certain pro-apoptotic peptides that bind to proteins expressed on the luminal side of capillaries) can gain access to these proteins because deoxycholate may cause vascular leakage. Thus, such agents can be synergistic with deoxycholate potentially creating a more potent means to mediate body contouring in fewer therapeutic sessions.

Examples of NPY antagonists include, but are not limited to, NPY receptor antagonists, such as BIBP-3226 (Amgen), BIBO-3304 (Boehringer Ingleheim), BMS-192548 and AR-H040922 (Bristol-Myers Squibb), LY-357897 (Eli Lilly), 1229U91 and GW4380145 (GlaxoSmithKline), JNJ-5207787 (Johnson & Johnson), Lu-AA-44608 (Lundbeck), MK-0557 (Merck NPY), NGD-95-1 (Neurogen), NLX-E201 (Neurologix), CGP-71683 (Novartis), PD-160170 (Pfizer), SR-120819A, BIIE0246, and S.A. 0204 (Sanofi Aventis), S-2367 (Shiongli), dihydropyridine and dihydropyridine derivatives that are NPY receptor antagonists, bicyclic compounds that are NPY receptor antagonists, carbazole NPY receptor antagonists, and tricyclic compounds that are NPY receptor antagonists. See, e.g., WO 2006/133160 and U.S. Pat. No. 6,313,128.

Exemplary fat selective pro-apoptotic peptides includes, but is not limited to, CKGGRAKDC peptide that homes to white fat vasculature. See, Kolonin M. G. et al., Nat. Med. June 10(6):625-32 (2004).

Sodium deoxycholate or sodium (4R)-4-((3R,5R,10S,12S,13R,17R)-3,12-dihydroxy-10,13-dimethylhexadecahydro-1H-cyclopenta[a]phenanthren-17-yl)pentanoate can be prepared according to the methods disclosed in PCT/US2010/061150 titled “Methods for the Purification of Deoxycholic Acid,” filed on Dec. 17, 2010. Other salts of deoxycholic acid can be prepared likewise by the skilled artisan.

In one embodiment of this invention, the formulation of deoxycholic acid salt utilized is substantially stabilized against precipitation over a period of time preferably for at least about six months. In another aspect, the methods stabilize the formulation of deoxycholic acid salt against precipitation for a period of at least about one year. In yet another aspect, the methods stabilize the formulation of deoxycholic acid salt against precipitation for a period of at least about two years.

It has been found that the pH of the solution can inhibit the precipitation of deoxycholic acid or a salt thereof at concentrations of from about 0.4% w/v to less than about 2% w/v in water to allow deoxycholic acid or a salt thereof, to be maintained in solution. In one embodiment, the pH is established by the use of a base. It is contemplated that any base can be used to increase the pH of the composition provided that it does not react with deoxycholic acid or a salt thereof. In some embodiments, the base is selected from the group consisting of metal carbonates, metal bicarbonates, and metal hydroxides, or a mixture thereof. Examples of bases include, but are not limited to, a base selected from the group consisting of sodium carbonate, calcium carbonate, magnesium carbonate, zinc carbonate, sodium bicarbonate, sodium hydroxide and potassium hydroxide or a mixture thereof. In one embodiment, the base is sodium hydroxide.

In certain embodiments, the pH ranges from about 8.1 to about 8.5. In one embodiment, the pH of the composition is about 8.1, or alternatively, about 8.2, or alternatively, about 8.3, or alternatively, about 8.4, or alternatively, about 8.5. In a preferred embodiment, the pH of the aqueous solution is about 8.3.

In certain cases, the pH of the composition may need to be maintained with the use of a buffer. Various buffers are known in the art and it is contemplated that any buffer having buffering capacity at the desired pH can be used in the formulations disclosed herein. In one embodiment, the buffer is a phosphate buffer. The amount of phosphate required to provide a desired pH and salt concentration can be calculated using methods well known in the art. In one embodiment, the composition comprises about 10 mM phosphate buffer. In another embodiment, the phosphate buffer is 10 mM dibasic sodium phosphate buffer.

In certain cases, the pH is established by use of a base and optionally maintained by use of a buffer.

In one embodiment, the methods utilized herein provide formulations which are suitable for injection into a human. The method of injection can be any type of injection, such as subcutaneous injection, as well as other forms of injection. Therefore, in some embodiments, the aqueous solution comprises sterile water or water for injection (WFI).

In one aspect, it may be that one or more excipients are used to maintain the solubility, or increase the preservability of deoxycholic acid salt present in the formulation. In one embodiment, the method comprises adding about 1% w/v benzyl alcohol. In some embodiments, the formulation also comprises at least one excipient to aid in achieving an isotonic solution. Such excipients are known in the art. In one embodiment, the method comprises adding about 1% w/v sodium chloride. In some embodiments, the method comprises adding both 1% w/v benzyl alcohol and 1% w/v sodium chloride. In some embodiments, the method comprises adding both 0.9% w/v benzyl alcohol and 0.9% w/v sodium chloride. Using the methods disclosed herein, an aqueous solution comprising less than about 2% w/v of deoxycholic acid salt is maintained at a pH sufficient to substantially inhibit precipitation of deoxycholic acid salt. The amount of precipitation or homogeneity of the composition can be measured using various methods. For example, it can be measured quantitatively by measuring the light scattering via illumination by a spectrophotometer. Or alternatively, the homogeneity can be measured qualitatively by simply observing the visual clarity of the solution with the eye. In some embodiments, the method provides a pharmaceutical composition having a relative standard deviation for homogeneity of less than about 5%. Alternatively, the relative standard deviation for homogeneity of less than about 4%, or alternatively, about 3%, or alternatively, about 2%, or alternatively, about 1%.

The storage temperature can assist in maintaining the solubility of deoxycholic acid salt in the formulation. In certain embodiments, the storage temperature is from about 17° C. to about 27° C. In some embodiments, the storage temperature is about 25° C. to about 37° C. In other embodiments, the storage temperature is from about 2° C. to about 8° C.

It is contemplated that the concentration of the salt of deoxycholic acid in the formulation is about 0.5% w/v, or alternatively about 0.7% w/v, or alternatively about 1% w/v, or alternatively about 1.2% w/v, or alternatively about 1.4% w/v, or alternatively less than about 2% w/v. In a preferred embodiment, the salt of deoxycholic acid is sodium deoxycholate. In another preferred embodiment, the composition comprises 0.5% w/v of sodium deoxycholate. In another preferred embodiment, the composition comprises 1% w/v of sodium deoxycholate.

In one embodiment, the aqueous formulation is split into a plurality of individual solutions which are separately administered to the fat cells. For example, the aqueous formulation is split into 5, 10, 15, 20, 25 or 30 separate solutions and, in some cases, up to 50 separate solutions.

In a preferred embodiment, the salt of deoxycholic acid is sodium deoxycholate. As the methods of this invention include subcutaneous injections, there is also provided a syringe comprising a chamber, a plunger and an injection needle wherein the chamber comprises a formulation of this invention. Preferably, the chamber is sufficient to hold at least 2 mL and preferably no more than 4 mL of the formulation.

In another embodiment, this invention provides a synthesis of DCA from protected commercially available 9-α,17-β-dihydroxy-5-α-androstan-3-one as shown in scheme 1 below.

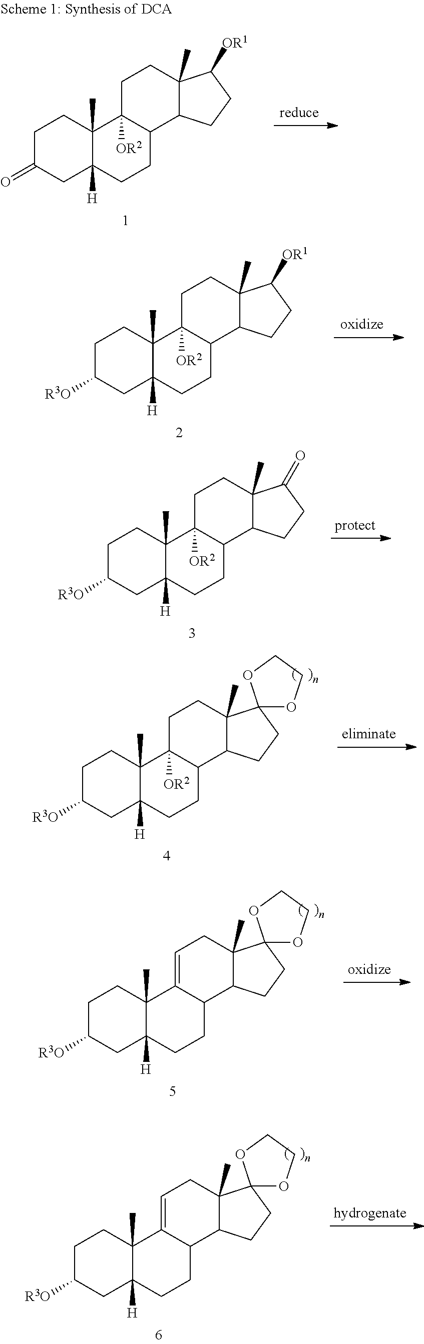

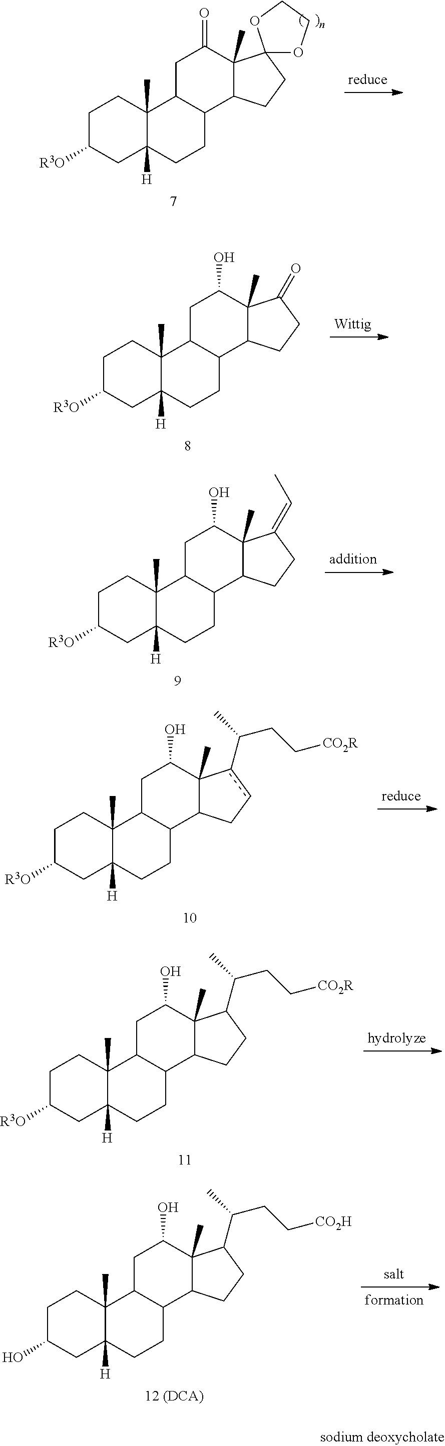

The 9-α,17-β hydroxyl groups of commercially available 9-α,17-β-dihydroxy-5-α-androstan-3-one are differentially protected with hydroxyl protecting groups which can be removed under conditions where one of the hydroxyl groups is regenerated while the other hydroxyl group remains protected. Such differential protection is referred to as orthogonal protection and uses well known reagents and reaction conditions. In one example, one hydroxy group is protected as an acetyl group, whereas the other hydroxy group is protected as a benzyl group. Each group can be selectively removed under reaction conditions that retain the other hydroxyl protecting group intact.

It is contemplated that the relatively sterically protected 9-α-hydroxyl group may not need to be protected as the reactions contemplated prior to elimination of that group are likely inhibited at this position due to steric hindrance. Regardless, protection of this hydroxyl group adequately insures that the group remains intact until elimination of the hydroxyl group via dehydration is desired.

The 3-one group of orthogonally protected 9-α,17-β-dihydroxy-5-α-androstan-3-one, compound 1, is reduced with conventional reducing agent such as sodium borohydride to provide the 3-α-hydroxy derivative which is then protected with yet another orthogonal protecting group to provide compound 2.

The hydroxyl protecting group at the 17-position of compound 2 is then selectively removed and the hydroxyl group so regenerated is then oxidized with a suitable oxidation reagent such as CrO₃ to provide the 17-keto derivative, compound 3. The 17-keto group in compound 3 is protected as a ketal under standard ketalization conditions such as reaction with 1,2-dihydroxyethane or 1,3-dihydroxypropane to give compound 4 (which illustrates ketal formation with the 1,2-dihydroxyethane for illustrative purposes only).

Deprotection of the 9-α-hydroxyl as necessary is followed by dehydration of that hydroxyl group under conditions such as acid-catalyzed elimination provides the 9,10-unsaturated derivative, compound 5. Generation of a 12-keto group is accomplished by allylic oxidation of compound 5 with oxidation reagents such as chromic acid or TBHP (tert-butyl hydroperoxide) and NaOCl to provide compound 6. See, for example, U.S. Patent Application Ser. No. 61/348,686. Alternatively, the allylic oxidation is accomplished by using about 2 to 5 equivalents of TBHP and about 0.3 to 0.5 equivalents of CuI as a catalyst. The reaction is carried out in a solvent such as acetonitrile at 40° C. for about 40-55 hours. The slow portionwise addition of TBHP results in more efficient oxidation. The product formed contains a mixture of compound 6 and the corresponding allylic alcohol. The product mixture is then oxidized with PCC to give compound 6.

Hydrogenation of compound 6 under standard conditions such as 10% Pd/C and H₂ provides compound 7. Reduction of the 12-keto group in compound 7 with reagents such as LiAl(OBu^{t})₃H provides the 12-hydroxy derivative, compound 8. Olefination of compound 8 under standard Wittig conditions such as using ethyltriphenylphosphonium bromide in presence of a base such as potassium tert-butoxide provides compound 9. Addition of an alkyl acrylate such as methyl acrylate in presence of a Lewis acid provides compound 10, wherein R is an alkyl group such as methyl. Reduction of the double bond in compound 10 again proceeds under standard hydrogenation conditions such as Pd/C and H₂ to provide compound 11. Deprotection of the 3-OR₃ followed by hydrolysis with a base such as LiOH provides DCA, compound 12.

Compound 12 (crude DCA) was further purified with methanol wash and recrystallized from ethanol. It was diluted with 2 mol % MeOH in CH₂Cl₂ (25 vol) and heated to 35-37° C. for 1 hour. The slurry was allowed to cool to 28-30° C. and filtered. The filter cake was washed with CH₂Cl₂ (5 vol) and dried under vacuum at 40° C. to afford DCA.

DCA was dissolved in 10% DI water/EtOH (12 vol), polish filtered over celite and washed with 10% DI water/EtOH (3 vol). The resulting 15 volume filtrate was added to DI water (30 vol) and a thin white slurry was afforded. The slurry was held for 24 hours, filtered, washed with DI water (20 vol) and dried under vacuum at 40° C. to afford DCA.

Conversion of DCA to a pharmaceutically acceptable salt such as sodium deoxycholate proceeds via conventional conditions. Alternatively, conversion of a pharmaceutically acceptable salt of DCA such as sodium deoxycholate to DCA also proceeds via conventional conditions.

In another embodiment, this invention utilizes a stabilized formulation comprising:

a buffered aqueous solution having a pH of about 8.1 to about 8.5 and further

comprising about 0.5% of sodium deoxycholate and about 0.9% of benzyl alcohol, wherein the formulation is stabilized against precipitation, and the sodium deoxycholate is prepared according to scheme 1.

In another embodiment, this invention utilizes a stabilized formulation comprising:

a buffered aqueous solution having a pH of about 8.1 to about 8.5 and further

comprising about 1% of sodium deoxycholate and about 0.9% of benzyl alcohol, wherein the formulation is stabilized against precipitation, and the sodium deoxycholate is prepared according to scheme 1.

Deoxycholic acid, or a salt thereof, or any composition thereof described herein may be used for any of the methods disclosed herein.

### Methods

In one aspect, provided herein is a method of treating accumulated fat in a patient in need thereof, comprising administering into the accumulated fat a therapeutically effective amount of DCA or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea. In some embodiments, the administering step is conducted by subcutaneous injection.

In another aspect, provided herein is a method of treating accumulated fat in a patient in need thereof, comprising administering into the accumulated fat a therapeutically effective amount of DCA or a pharmaceutically acceptable salt thereof and at least one pharmaceutically acceptable excipient, wherein the accumulated fat is one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, and post-liposuction fat deposits. In some embodiments, the administering step is conducted by subcutaneous injection.

In another aspect, provided herein is a method of reducing accumulated fat in a patient in need thereof, comprising administering into the accumulated fat a therapeutically effective amount of DCA or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea. In some embodiments, the administering step is conducted by subcutaneous injection.

In another aspect, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes bra fat (including, but not limited to, one or more of excess axillary fat, lateral periaxillary fat, pre axillary fat, post axillary fat, anterior periaxillary fat, posterior periaxillary fat, and fat on the upper back,). In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes excess axillary fat. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes periaxillary fat. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes lateral periaxillary fat. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes pre axillary fat. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes post axillary fat. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes fat on the upper back. In some embodiments, the administering step is conducted by subcutaneous injection.

In some embodiments, provided herein is a method of reducing bra fat in a patient in need thereof, comprising locally administering into the bra fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing excess axillary fat in a patient in need thereof, comprising locally administering into the excess axillary fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing periaxillary fat in a patient in need thereof, comprising locally administering into the periaxillary fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing lateral periaxillary fat in a patient in need thereof, comprising locally administering into the lateral periaxillary fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing pre axillary fat in a patient in need thereof, comprising locally administering into the pre axillary fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing post axillary fat in a patient in need thereof, comprising locally administering into the post axillary fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing posterior periaxillary fat in a patient in need thereof, comprising locally administering into the posterior periaxillary fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing anterior periaxillary axillary fat in a patient in need thereof, comprising locally administering into the anterior periaxillary axillary fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing fat on the upper back in a patient in need thereof, comprising locally administering into the fat on the upper back a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes back of arm fat. In some embodiments, the administering step is conducted by subcutaneous injection.

In some embodiments, provided herein is a method of reducing back of arm fat in a patient in need thereof, comprising locally administering into the back of arm fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes a love handle. In some embodiments, the administering step is conducted by subcutaneous injection.

In some embodiments, provided herein is a method of reducing a love handle in a patient in need thereof, comprising locally administering into the love handle a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes fat on the anterolateral flank. In some embodiments, the administering step is conducted by subcutaneous injection.

In some embodiments, provided herein is a method of reducing fat on the anterolateral flank in a patient in need thereof, comprising locally administering into the fat on the anterolateral flank a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes excess fat at the sides of the waistline of the patient. In some embodiments, the administering step is conducted by subcutaneous injection.

In some embodiments, provided herein is a method of reducing deposits of excess fat at the sides of the waistline in a patient in need thereof, comprising locally administering into the deposits of excess fat at the sides of the waistline a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes medial knee fat. In some embodiments, the administering step is conducted by subcutaneous injection.

In some embodiments, provided herein is a method of reducing medial knee fat in a patient in need thereof, comprising locally administering into the medial knee fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes inner upper thigh fat. In some embodiments, the administering step is conducted by subcutaneous injection.

In some embodiments, provided herein is a method of reducing inner upper thigh fat in a patient in need thereof, comprising locally administering into the inner upper thigh fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes outer upper thigh fat. In some embodiments, the administering step is conducted by subcutaneous injection.

In some embodiments, provided herein is a method of reducing outer upper thigh fat in a patient in need thereof, comprising locally administering into the outer upper thigh fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes calf fat. In some embodiments, the administering step is conducted by subcutaneous injection.

In some embodiments, provided herein is a method of reducing calf fat in a patient in need thereof, comprising locally administering into the calf fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes fat around the ankles. In some embodiments, the administering step is conducted by subcutaneous injection.

In some embodiments, provided herein is a method of reducing fat around the ankles in a patient in need thereof, comprising locally administering into the fat around the ankles a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes excess fat on the face. In some embodiments, the administering step is conducted by subcutaneous injection.

In some embodiments, provided herein is a method of reducing excess fat on the face in a patient in need thereof, comprising locally administering into the excess fat on the face a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes intraorbital fat. In some embodiments, the administering step is conducted by subcutaneous injection.

In some embodiments, provided herein is a method of reducing intraorbital fat in a patient in need thereof, comprising locally administering into the intraorbital fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes periorbital fat. In some embodiments, the administering step is conducted by subcutaneous injection.

In some embodiments, provided herein is a method of reducing periorbital fat in a patient in need thereof, comprising locally administering into the periorbital fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes malar fat and/or jaw fat. In some embodiments, the administering step is conducted by subcutaneous injection.

In some embodiments, provided herein is a method of reducing malar fat and/or jaw fat in a patient in need thereof, comprising locally administering into the malar fat and/or jaw fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes stomach fat (including, but not limited to periumbilical fat, fat above periumbilical area, fat below periumbilical area, or any combination of two or more thereof). In some embodiments, the administering step is conducted by subcutaneous injection. In some embodiments, stomach fat is referred to as fat above and below periumbilical area. As used herein, the term “fat above and below periumbilical area” refers to fat above and below the belly button.

In some embodiments, provided herein is a method of reducing stomach fat in a patient in need thereof, comprising locally administering into the stomach fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection. In some embodiments, stomach fat is referred to as fat above and below periumbilical area.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes periumbilical fat. In some embodiments, the administering step is conducted by subcutaneous injection.

In some embodiments, provided herein is a method of reducing periumbilical fat in a patient in need thereof, comprising locally administering into the periumbilical fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes fat above periumbilical area. In some embodiments, the administering step is conducted by subcutaneous injection. As used herein, the term “fat above periumbilical area” refers to fat above the belly button or fat on the upper stomach.

In some embodiments, provided herein is a method of reducing fat above periumbilical area in a patient in need thereof, comprising locally administering into the fat above periumbilical area a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes fat below periumbilical area. In some embodiments, the administering step is conducted by subcutaneous injection. As used herein, the term “fat below periumbilical area” refers to fat below the belly button or fat on the lower stomach.

In some embodiments, provided herein is a method of reducing fat below periumbilical area in a patient in need thereof, comprising locally administering into the fat below periumbilical area a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes excess fat on the back or buttocks. In some embodiments, the administering step is conducted by subcutaneous injection.

In some embodiments, provided herein is a method of reducing excess fat on the back in a patient in need thereof, comprising locally administering into the excess fat on the back a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing excess fat on the buttocks in a patient in need thereof, comprising locally administering into the excess fat on buttocks a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes mons pubis fat. In some embodiments, the administering step is conducted by subcutaneous injection.

In some embodiments, provided herein is a method of reducing mons pubis fat in a patient in need thereof, comprising locally administering into the mons pubis fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes fat on the upper back of the thigh. In some embodiments, the administering step is conducted by subcutaneous injection.

In some embodiments, provided herein is a method of reducing fat on the upper back of the thigh in a patient in need thereof, comprising locally administering into the fat on the upper back of the thigh a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes excess fat on the foot. In some embodiments, the administering step is conducted by subcutaneous injection.

In some embodiments, provided herein is a method of reducing excess fat on the foot in a patient in need thereof, comprising locally administering into the excess fat on the foot a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes pseudogynocomastia fat. In some embodiments, the administering step is conducted by subcutaneous injection.

In some embodiments, provided herein is a method of reducing pseudogynocomastia fat in a patient in need thereof, comprising locally administering into the pseudogynocomastia fat a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes lipoma. In some embodiments, the administering step is conducted by subcutaneous injection.

In some embodiments, provided herein is a method of reducing a lipoma in a patient in need thereof, comprising locally administering into the lipoma a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes lipodystrophy (such as Dunning-type lipodystrophy). In some embodiments, the administering step is conducted by subcutaneous injection.

In some embodiments, provided herein is a method of treating lipodystrophy (such as Dunning-type lipodystrophy) in a patient in need thereof, comprising local administration of a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes lipomatosis such as familial multiple lipomatosis. In some embodiments, the administering step is conducted by subcutaneous injection.

In some embodiments, provided herein is a method of treating lipomatosis such as familial multiple lipomatosis in a patient in need thereof, comprising local administration of a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes post-liposuction fat deposits. In some embodiments, the administering step is conducted by subcutaneous injection.

In some embodiments, provided herein is a method of reducing post-liposuction fat deposits in a patient in need thereof, comprising locally administering into the post-liposuction fat deposits a composition comprising or consisting essentially of a therapeutically effective amount of DCA or a pharmaceutically acceptable salt thereof and at least one pharmaceutically acceptable excipient. In some embodiments, the administering step is conducted by subcutaneous injection.

In some embodiments, provided herein is a method of reducing accumulated fat in a patient in need thereof, comprising administering into the accumulated fat a composition comprising or consisting essentially of a therapeutically effective amount of DCA or a pharmaceutically acceptable salt thereof and at least one pharmaceutically acceptable excipient, wherein the accumulated fat results from or causes obstructive sleep apnea. In some embodiments, the administering step is conducted by subcutaneous injection.

In another aspect, provided herein is a method of reducing accumulated fat in a patient in need thereof, comprising administering into the accumulated fat by local injection a composition comprising or consisting essentially of a therapeutically effective amount of DCA or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea. In some embodiments, the administering step is conducted by subcutaneous injection.

In another aspect, provided herein is a method of reducing accumulated fat in a patient in need thereof, comprising administering into the accumulated fat by local subcutaneous injection a composition comprising or consisting essentially of a therapeutically effective amount of DCA or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea.

In another aspect, provided herein is a method of reducing accumulated fat in a patient in need thereof, comprising administering into the accumulated fat by a plurality of injections (i.e., two or more injections) a composition comprising or consisting essentially of a therapeutically effective amount of DCA or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea.

In another aspect, provided herein is a cosmetic method of reducing accumulated fat in a subject in need thereof, comprising administering into the accumulated fat by subcutaneous injection a composition comprising or consisting essentially of DCA, or a salt thereof, and at least one cosmetically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the back or buttocks, mons pubis fat, excess fat around the ankles, fat on the upper back of the thigh, excess fat on the foot, pseudogynocomastia fat, lipoma, lipomatosis such as familial multiple lipomatosis, and post-liposuction fat deposits.

In another aspect, provided herein is a method of correcting one or more post-interventional treatment contour irregularities in a subject in need thereof, comprising administering to the subject a composition described herein. Post-interventional treatment contour irregularities include, but are not limited to, contour irregularities following one or more of plastic surgery, breast augmentation, reconstruction, mammopexy, fat injection, liposuction, energy-based treatment (including, but not limited to cryotherapy, radio frequency treatment, laser treatment), and breast reduction (male or female).

In another aspect, provided herein is a method of correcting one or more post-interventional treatment contour irregularities in a subject in need thereof, comprising administering to the subject a composition comprising or consisting essentially of a therapeutically effective amount of DCA or a pharmaceutically acceptable salt thereof and at least one pharmaceutically acceptable or cosmetically acceptable excipient.

In another aspect, provided herein is a method of correcting one or more post-interventional treatment contour irregularities in a subject in need thereof, comprising administering to the subject a composition comprising or consisting essentially of a therapeutically effective amount of DCA or a pharmaceutically acceptable salt thereof and at least one pharmaceutically acceptable or cosmetically acceptable excipient, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

In another aspect, provided herein is a method of correcting one or more post-interventional treatment contour irregularities in a subject in need thereof, comprising locally administering to the subject a composition comprising or consisting essentially of a therapeutically effective amount of DCA or a pharmaceutically acceptable salt thereof and at least one pharmaceutically acceptable or cosmetically acceptable excipient, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

In another aspect, provided herein is a method of correcting one or more post-interventional treatment contour irregularities in a subject in need thereof, comprising locally administering to the subject by one or more subcutaneous injections a composition comprising or consisting essentially of a therapeutically effective amount of DCA or a pharmaceutically acceptable salt thereof and at least one pharmaceutically acceptable or cosmetically acceptable excipient, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

In another aspect, provided herein is a method of correcting one or more post-interventional treatment contour irregularities in a subject in need thereof, comprising locally administering to the subject by a plurality of subcutaneous injections a composition comprising or consisting essentially of a therapeutically effective amount of DCA or a pharmaceutically acceptable salt thereof and at least one pharmaceutically acceptable or cosmetically acceptable excipient, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

In another aspect, provided herein is a cosmetic method of correcting one or more post-interventional treatment contour irregularities in a subject in need thereof, comprising locally administering to the subject a composition comprising or consisting essentially of an effective amount of DCA or a pharmaceutically acceptable salt thereof and at least one cosmetically acceptable excipient, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

In another aspect, provided herein is a cosmetic method of correcting one or more post-interventional treatment contour irregularities in a subject in need thereof, comprising locally administering to the subject by one or more subcutaneous injections a composition comprising or consisting essentially of an effective amount of DCA or a pharmaceutically acceptable salt thereof and at least one cosmetically acceptable excipient, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

In another aspect, provided herein is a cosmetic method of correcting one or more post-interventional treatment contour irregularities in a subject in need thereof, comprising locally administering to the subject by a plurality of subcutaneous injections a composition comprising or consisting essentially of an effective amount of DCA or a pharmaceutically acceptable salt thereof and at least one cosmetically acceptable excipient, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

The accumulated fat is treated and/or reduced by administering the DCA or a salt thereof into the accumulated fat. The accumulated fat to be treated and/or reduced can be divided into small grids, such as into a grid 1.5 cm apart. See, e.g., US 2012/0237492, incorporated herein in its entirety by reference. DCA or a salt thereof, formulated as described herein, or in other ways well known in the art is administered into each grid. The accumulated fat may be pulled away from the underlying structure, e.g., and without limitation, a pinch and pull technique, before administering the DCA or the salt thereof. Various injection techniques can be used including, without limitation, the use of a syringe, the use of multi-injector device such as mesorelle, and the use of mesogun.

In some embodiments, the compositions described herein are administered as 0.2 mL injections spaced 1-cm apart. In some embodiments, up to 50 injections or 10 mL is injected in a single treatment. In some embodiments, up to 6 single treatments are administered at intervals of no less than 1 month apart. In some embodiments, the compositions described herein are administered as 0.2 mL injections spaced 1-cm apart; up to 50 injections or 10 mL is injected in a single treatment; and up to 6 single treatments are administered at intervals of no less than 1 month apart.

### EXAMPLES

In the examples and elsewhere in the specification, abbreviations have the following meanings:
mg=milligram
mL=milliliter
mm=millimeter
mM=millimolar
T=Time
UV=Ultraviolet
v/v=Volume/Volume
w/v=Weight/Volume (g/mL)
w/w=Weight/Weight
WFI=Water for Injection
mOsm=Milliosmole

The invention is further understood by reference to the following examples, which are intended to be purely exemplary of the invention.

### Example 1. Concentration Dependent Precipitation from a Solution of Sodium Deoxycholate

Solutions of sodium deoxycholate at different concentration were evaluated for precipitate formation after 1 week of storage. The results demonstrate that at about 0.5% and at about 1% (w/v) concentration of sodium deoxycholate in an aqueous solution containing only water and 0.9% w/v benzyl alcohol, a significant amount of precipitate is formed such that it would inhibit use of the solution as a composition for subcutaneous injections. By visual inspection, the amount of precipitation can be rated as tabulated below.

TABLE 1

| % w/v Sodium | Precipitation | |
|---|---|---|
| Deoxycholate | Rating | Comment |
| 0 | 1 | Precipitation substantially invisible to the |
| | | naked eye |
| 0.5 | 10 | Significant amounts of precipitate visible |
| | | to the naked eye |
| 1.0 | 7 | Significant amounts of precipitate visible |
| | | to the naked eye but less than that for |
| | | 0.5% w/v |
| 2.0 | 2 | Precipitation visible to the naked eye but |
| | | present in substantially smaller amounts |
| | | compared to the 0.5 and 1% solutions |
| | | above |

The precipitation rating estimates that “0” refers a clear solution and that “10” refers to a mixture exhibiting substantial precipitation readily visible to the naked eye.

Such an observation demonstrates that in the concentration ranges tested, the precipitation phenomena was substantially affected by deoxycholate concentration. To ascertain pH's effect on precipitation, the pH of the solutions were measured, as provided in Table 2, which demonstrates that the pH of the solutions were substantially the same, especially for the 1% and the 2% solutions. The inverse aqueous solubility of sodium deoxycholate, where a more dilute solution (0.5% or 1%) provides more precipitation than a more concentrated solution (2%), is a surprising observation and also evidences that the precipitation phenomena is not directly related to pH, because the pH of the solutions were substantially the same again, especially for the 1% and 2% solutions.

TABLE 2

| % w/v Sodium | | | |
|---|---|---|---|
| Deoxycholate | Reading # | Temperature/° C. | pH |
| 0 | 1 | 24.0 | 7.75 |
| 0 | 2 | 24.1 | 7.58 |
| 0.5 | 1 | 24.7 | 7.77 |
| 0.5 | 2 | 24.5 | 7.71 |
| 1 | 1 | 24.6 | 7.93 |
| 1 | 2 | 24.5 | 7.97 |
| 2 | 1 | 24.9 | 8.07 |
| 2 | 2 | 24.7 | 8.06 |

Accordingly, this invention provides that the surprising precipitation from dilute, 0.4% to less than 2% (w/v), salt of deoxycholic acid solutions are inhibited, to the extent that such solutions are useful for subcutaneous injections, by increasing the solution pH.

### Example 2. Sodium Deoxycholate (API) Formulations with and without Benzyl Alcohol

1. A composition of sodium deoxycholate (0.5% and 1%) was prepared comprising sodium phosphate (10 mM), sodium chloride (75-90 mM), benzyl alcohol (0.9%), deoxycholic acid, pH 8.3.

2. An isotonic composition of sodium deoxycholate without benzyl alcohol was prepared using the free acid form, namely, deoxycholic acid, as follows.

a. Preparation of 100 mL Isotonic Batches at 10 mg/mL

1.0 g of the deoxycholic acid (DCA) was added to the solution only after a basic solution was made with 70 mL water, 142 mg anhydrous dibasic sodium phosphate and 267 μL 10M NaOH. It took about 20 minutes for the API to go into solution. The pH of the solution was 11.1. The rapid addition of HCl was known to cause some precipitation, so 225 μL of 1M HCl was slowly added to bring the solution to pH 8.3. The solution was allowed to mix for an additional 15 minutes. After bringing the volume up to 100 mL with water, the osmolality was found to be 51 mOsm. Addition of 859 mg of NaCl brought the osmolality up to 305 mOsm.

The solution so prepared could optionally be lyophilized to provide for a lyophilized product which could be reconstituted by addition of the appropriate amount of sterile water. Accordingly, this invention also provides for lyophilized products of the solutions disclosed herein.

b. Preparation of 1000 mL Isotonic Batches at 10 mg/mL

The results from section a (above) did not scale up perfectly when multiplied ten fold. To 900 mL of water, 1.4 g anhydrous dibasic sodium phosphate, 8.6 g NaCl, and 2.7 mL 10 M NaOH were added. 10.0 g of DCA was then added and allowed to mix to clarity for 30 minutes. The pH of the solution was 10.4. 1.5 mL 1 M HCl was slowly added and allowed to mix for 5 minutes. The final pH was 8.1. An additional 20 μL of 10M NaOH had to be added to bring the pH to 8.3. After bringing the volume up to 1000 mL with water, the osmolality was 314 mOsm.

Based on observations of the pH change during the addition of 1 M HCl, it was determined that for 1000 mL batches at 10 mg/mL API, just 1.0 mL of 1M HCl should be immediately added and then slowly titrated with small volumes of the acid. The suggested order of addition for 1000 mL of 10 mg/mL API is outlined in Table 3.

c. Preparation of 100 mL Isotonic Batches at 5 mg/mL

0.50 g of deoxycholic acid (DCA) was added to the solution only after a basic solution was made with 70 mL water, 142 mg anhydrous dibasic sodium phosphate and 134 μL 10M NaOH. It took about 20 minutes for the API to go into solution. The pH was 10.7. The rapid addition of HCl was known to cause some precipitation, so 115 μL of 1 M HCl was slowly added to bring the solution to pH 8.3. The solution was allowed to mix for an additional 15 minutes. After bringing the volume up to 100 mL with water, the osmolality was found to be 39 mOsm. Addition of 859 mg of NaCl brought the osmolality up to 294 mOsm.

d. Preparation of 1000 mL Isotonic Batches at 5 mg/mL

The results from section c (above) did not scale up perfectly when multiplied ten fold. To 900 mL of water, 1.4 g anhydrous dibasic sodium phosphate, 8.6 g NaCl, and 1.3 mL 10M NaOH were added. 5.0 g of DCA was then added and allowed to mix to clarity for 30 minutes. The pH was 8.6. After adding just 350 μL 1M HCl, the pH dropped to 8.0. An additional 25 μL of 10M NaOH had to be added to bring the pH to 8.4. After bringing the volume up to 1000 mL with water, the osmolality was 305 mOsm. Based on observations of the pH change during the addition of 1 M HCl, it was determined that for 1000 mL batches at 5 mg/mL API, that the solution should be slowly titrated with small volumes of 1M HCl. The suggested order of addition for 1000 mL of 5 mg/mL is outlined in Table 3.

TABLE 3
Order of addition (left to right) for isotonic 1000 mL
benzyl alcohol free formulation

| | Dibasic | | | | | |
|---|---|---|---|---|---|---|
| | Anhydrous | | | | | |
| API | Sodium | 10M | | | | |
| Concentration | Phosphate | NaOH | DCA | NaCl | 1M HCl | pH |
| 10 mg/mL | 1.4 g | 2.7 mL | 10.0 g | 8.6 g | 1.0 mL + | 8.3 |
| | | | | | incremental | |
| | | | | | addition to | |
| | | | | | final pH | |
| 5 mg/mL | 1.4 g | 1.3 mL | 5.0 g | 8.6 g | incremental | 8.3 |
| | | | | | addition to | |
| | | | | | final pH | |

### Example 3. Treatment of Bra Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes bra fat.

### Example 4. Treatment of Back of Arm Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes back of arm fat.

### Example 5. Treatment of a Love Handle

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes a love handle.

### Example 6. Treatment of Back of Medial Knee Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes back of medial knee fat.

### Example 7. Treatment of Inner Upper Thigh Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes inner upper thigh fat.

### Example 8. Treatment of Outer Upper Thigh Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes outer upper thigh fat.

### Example 9. Treatment of Back of Calf Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes calf fat.

### Example 10. Treatment of Fat Around the Ankles

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes fat around the ankles.

### Example 11. Treatment of Excess Fat on the Face

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes excess fat on the face.

### Example 12. Treatment of Intraorbital Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes intraorbital fat.

### Example 13. Treatment of Periorbital Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes periorbital fat.

### Example 14. Treatment of Malar Fat and/or Jaw Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes malar fat and/or jaw fat.

### Example 15. Treatment of Stomach Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes stomach fat.

### Example 16. Treatment of Periumbilical Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes periumbilical fat.

### Example 17. Treatment of Excess Fat on the Back

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes excess fat on the back.

### Example 18. Treatment of Excess Fat on the Buttocks

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes excess fat on the buttocks.

### Example 19. Treatment of Mons Pubis Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes mons pubis fat.

### Example 20. Treatment of Lipoma

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes lipoma.

### Example 21. Treatment of Lipodystrophy

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes lipodystrophy.

### Example 22. Treatment of Lipomatosis

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes lipomatosis.

### Example 23. Treatment of Post-Liposuction Fat Deposits

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes post-liposuction fat deposits.

### Example 24. Treatment of Deposits of Excess Fat at the Sides of the Waistline

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes deposits of excess fat at the sides of the waistline.

### Example 25. Treatment of Fat on the Anterolateral Flank

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes fat on the anterolateral flank.

### Example 26. Treatment of Excess Axillary Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes excess axillary fat.

### Example 27. Treatment of Periaxillary Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes periaxillary fat.

### Example 28. Treatment of Lateral Periaxillary Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes lateral periaxillary fat.

### Example 29. Treatment of Pre Axillary Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes pre axillary fat.

### Example 30. Treatment of Post Axillary Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes post axillary fat.

### Example 31. Treatment of Fat on the Upper Back

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes fat on the upper back.

### Example 32. Treatment of Fat on the Upper Back of the Thigh

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes fat on the upper back of the thigh.

### Example 33. Treatment of Excess Fat on the Foot

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes excess fat on the foot.

### Example 34. Treatment of Fat Above Periumbilical Area

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes fat above periumbilical area.

### Example 35. Treatment of Fat Below Periumbilical Area

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes fat below periumbilical area.

### Example 36. Treatment of Anterior Periaxillary Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes anterior periaxillary fat.

### Example 37. Treatment of Posterior Periaxillary Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes posterior periaxillary fat.

### Example 38. Treatment of Pseudogynocomastia Fat

An aqueous formulation described herein (e.g., 1% w/v deoxycholic acid, 0.9% w/v benzyl alcohol, 0.14% w/v dibasic sodium phosphate, 0.14% w/v sodium hydroxide, 0.44% w/v sodium chloride, and hydrogen chloride (q.s.); or 1% w/v deoxycholic acid, 0.14% w/v dibasic sodium phosphate, 0.14% w/v sodium hydroxide, 0.75% w/v sodium chloride, and hydrogen chloride (q.s.)) is locally administered as one or more subcutaneous injections to a human subject for reducing accumulated fat that results from or causes pseudogynocomastia fat.

Para. A. A method of treating accumulated fat in a patient in need thereof, comprising administering into the accumulated fat a therapeutically effective amount of deoxycholic acid (DCA) or a pharmaceutically acceptable salt thereof and at least one pharmaceutically acceptable excipient, wherein the accumulated fat results from or causes one or more of bra fat, back of arm fat, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including periumbilical fat), excess fat on the back or buttocks, mons pubis fat, excess fat around the ankles, lipoma, lipodystrophy (such as Dunning-type lipodystrophy), lipomatosis such as familial multiple lipomatosis, post-liposuction fat deposits, and obstructive sleep apnea.

Para. B. A method of reducing accumulated fat in a patient in need thereof, comprising administering into the accumulated fat a therapeutically effective amount of deoxycholic acid (DCA) or a pharmaceutically acceptable salt thereof and at least one pharmaceutically acceptable excipient, wherein the accumulated fat results from or causes one or more of bra fat, back of arm fat, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including periumbilical fat), excess fat on the back or buttocks, mons pubis fat, excess fat around the ankles, lipoma, lipodystrophy (such as Dunning-type lipodystrophy), lipomatosis such as familial multiple lipomatosis, post-liposuction fat deposits, and obstructive sleep apnea.

Para. C. The method of Para. A or Para. B, wherein the DCA is administered as a precipitation stable aqueous composition consisting essentially of from about 0.4% w/v to less than about 2% w/v of a salt of deoxycholic acid, wherein said composition is maintained at a pH of about 8.1 to about 8.5.

Para. D. The method of Para. A or Para. B, wherein the DCA is present in an amount from about 0.5% w/v to about 1% w/v.

Para. E. The method of Para. A or Para. B, wherein the DCA is present in an amount of about 0.5% w/v.

Para. F. The method of Para. A or Para. B, wherein the DCA is present in an amount of about 1% w/v.

Para. G. The method of Para. A or Para. B, wherein the excipient is a solvent, a buffer, a preservative, a lyophilization aid, or any combination thereof.

Para. H. The method of Para. A or Para. B, wherein the excipient is a solvent.

Para. I. The method of Para. A or Para. B, wherein the excipient is a preservative.

Para. J. The method of Para. A or Para. B, wherein said excipient is 0.9% benzyl alcohol.

Para. K. The method of any one of Paras. A to J, wherein said composition has a pH of about 8.3.

Para. L. The method of any one of Paras. A to K, wherein said salt is an alkali metal salt.

Para. M. The method of Para. L, wherein said alkali metal salt is sodium.

Para. N. The method of Para. A or Para. B, wherein the DCA is administered as a precipitation stable aqueous composition consisting essentially of:

a sterile aqueous solution buffered to a pH of about 8.3;

about 0.5% w/v of sodium deoxycholate;

about 0.9% w/v benzyl alcohol; and about 1% w/v of sodium chloride.

Para. O. The method of Para. A or Para. B, wherein the DCA is administered as a precipitation stable aqueous composition consisting essentially of:

an aqueous solution buffered to a pH of about 8.3;

about 1% w/v of sodium deoxycholate;

about 0.9% w/v benzyl alcohol; and

about 1% w/v of sodium chloride.

Para. P. The method of any one of Paras. A to 0, wherein the DCA is administered by injection.

Para. Q. The method of any one of Paras. A to 0, wherein the DCA is administered by a plurality of injections.

Para. R. Use of a composition comprising deoxycholic acid (DCA), or a pharmaceutically acceptable salt thereof, and at least one pharmaceutically acceptable excipient in a method of treating accumulated fat in a patient in need thereof by administering the composition to the patient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat lipoma, lipodystrophy (such as Dunning-type lipodystrophy), lipomatosis such as familial multiple lipomatosis, post-liposuction fat deposits, and obstructive sleep apnea.

Para. S. Use of a composition comprising deoxycholic acid (DCA), or a pharmaceutically acceptable salt thereof, and at least one pharmaceutically acceptable excipient in a method of reducing accumulated fat in a patient in need thereof by administering the composition to the patient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat lipoma, lipodystrophy (such as Dunning-type lipodystrophy), lipomatosis such as familial multiple lipomatosis, post-liposuction fat deposits, and obstructive sleep apnea.

Para. T. The use of Para. R or Para. S, wherein the composition is a precipitation stable aqueous composition consisting essentially of from about 0.4% w/v to less than about 2% w/v of a salt of deoxycholic acid, wherein said composition is maintained at a pH of about 8.1 to about 8.5.

Para. U. The use of Para. R or Para. S, wherein the DCA is present in an amount from about 0.5% w/v to about 1% w/v.

Para. V. The use of Para. R or Para. S, wherein the DCA is present in an amount of about 0.5% w/v.

Para. W. The use of Para. R or Para. S, wherein the DCA is present in an amount of about 1% w/v.

Para. X. The use of Para. R or Para. S, wherein the excipient is a solvent, a buffer, a preservative, a lyophilization aid, or any combination thereof.

Para. Y. The use of Para. R or Para. S, wherein the excipient is a solvent.

Para. Z. The use of Para. R or Para. S, wherein the excipient is a preservative.

Para. AA. The use of Para. R or Para. S, wherein said excipient is 0.9% benzyl alcohol.

Para. AB. The use of any one of Paras. R-AA, wherein said composition has a pH of about 8.3.

Para. AC. The use of any one of Paras. R-AB, wherein said salt is an alkali metal salt.

Para. AD. The use of Para. AC, wherein said alkali metal salt is sodium.

Para. AE. The use of Para. R or Para. S, wherein the composition consists essentially of:

a sterile aqueous solution buffered to a pH of about 8.3;

about 0.5% w/v of sodium deoxycholate;

about 0.9% w/v benzyl alcohol; and

about 1% w/v of sodium chloride.

Para. AF. The use of Para. R or Para. S, wherein the composition consists essentially of:

an aqueous solution buffered to a pH of about 8.3;

about 1% w/v of sodium deoxycholate;

about 0.9% w/v benzyl alcohol; and

about 1% w/v of sodium chloride.

Para. AG. The use of any one of Paras. R-AF, wherein the composition is administered by injection.

Para. AH. The use of any one of Paras. R-AF, wherein the composition is administered by a plurality of injections.

Para. AI. A method of treating accumulated fat in a patient in need thereof, comprising administering into the accumulated fat a therapeutically effective amount of deoxycholic acid (DCA) or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea.

Para. AJ. A method of reducing accumulated fat in a patient in need thereof, comprising administering into the accumulated fat a therapeutically effective amount of deoxycholic acid (DCA) or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea.

Para. AK. The method of Para. AI or Para. AJ, wherein the DCA is administered as a precipitation stable aqueous composition consisting essentially of from about 0.4% w/v to less than about 2% w/v of a salt of deoxycholic acid, wherein said composition is maintained at a pH of about 8.1 to about 8.5.

Para. AL. The method of Para. AI or Para. AJ, wherein the DCA is present in an amount from about 0.5% w/v to about 1% w/v.

Para. AM. The method of Para. AI or Para. AJ, wherein the DCA is present in an amount of about 0.5% w/v.

Para. AN. The method of Para. AI or Para. AJ, wherein the DCA is present in an amount of about 1% w/v.

Para. AO. The method of Para. AI or Para. AJ, wherein the excipient is a solvent, a buffer, a preservative, a lyophilization aid, or any combination thereof.

Para. AP. The method of Para. AI or Para. AJ, wherein the excipient is a solvent.

Para. AQ. The method of Para. AI or Para. AJ, wherein the excipient is a preservative.

Para. AR. The method of Para. AI or Para. AJ, wherein said excipient is 0.9% benzyl alcohol.

Para. AS. The method of any one of Paras. AI to AR, wherein said composition has a pH of about 8.3.

Para. AT. The method of any one of Paras. AI to AS, wherein said salt is an alkali metal salt.

Para. AU. The method of Para. AR, wherein said alkali metal salt is sodium.

Para. AV. The method of Para. AI or Para. AJ, wherein the DCA is administered as a precipitation stable aqueous composition consisting essentially of:

a sterile aqueous solution buffered to a pH of about 8.3;

about 0.5% w/v of sodium deoxycholate;

about 0.9% w/v benzyl alcohol; and

about 1% w/v of sodium chloride.

Para. AW. The method of Para. AI or Para. AJ, wherein the DCA is administered as a precipitation stable aqueous composition consisting essentially of:

an aqueous solution buffered to a pH of about 8.3;

about 1% w/v of sodium deoxycholate;

about 0.9% w/v benzyl alcohol; and

about 1% w/v of sodium chloride.

Para. AX. The method of any one of Paras. AI to AW, wherein the DCA is administered by injection.

Para. AY. The method of any one of Paras. AI to AW, wherein the DCA is administered by a plurality of injections.

Para. AZ. A method of correcting one or more post-interventional treatment contour irregularities in a subject in need thereof, comprising administering to the subject a composition comprising a therapeutically effective amount of DCA or a pharmaceutically acceptable salt thereof and at least one pharmaceutically acceptable or cosmetically acceptable excipient.

Para. BA. The method of Para. AZ, wherein the one or more contour irregularities result from plastic surgery, breast augmentation, reconstruction, mammopexy, fat injection, liposuction, energy-based treatment (including, but not limited to cryotherapy, radio frequency treatment, laser treatment), breast reduction (male or female), or a combination of two or more thereof.

Para. BB. The method of Para. AZ or Para. BA, wherein the DCA is administered as a precipitation stable aqueous composition consisting essentially of from about 0.4% w/v to less than about 2% w/v of a salt of deoxycholic acid, wherein said composition is maintained at a pH of about 8.1 to about 8.5.

Para. BC. The method of any one of Paras. AZ-BB, wherein the DCA is present in an amount from about 0.5% w/v to about 1% w/v.

Para. BD. The method of any one of Paras. AZ-BB, wherein the DCA is present in an amount of about 0.5% w/v.

Para. BE. The method of any one of Paras. AZ-BB, wherein the DCA is present in an amount of about 1% w/v.

Para. BF. The method of any one of Paras. AZ-BE, wherein the excipient is a solvent, a buffer, a preservative, a lyophilization aid, or any combination thereof.

Para. BG. The method of any one of Paras. AZ-BE, wherein the excipient is a solvent.

Para. BH. The method of any one of Paras. AZ-BE, wherein the excipient is a preservative.

Para. BI. The method of any one of Paras. AZ-BE, wherein said excipient is 0.9% benzyl alcohol.

Para. BJ. The method of any one of Paras. AZ-BI, wherein said composition has a pH of about 8.3.

Para. BK. The method of any one of Paras. AZ-BJ, wherein said salt is an alkali metal salt.

Para. BL. The method of Para. BK, wherein said alkali metal salt is sodium.

Para. BM. The method of Para. AZ, wherein the DCA is administered as a precipitation stable aqueous composition consisting essentially of:

a sterile aqueous solution buffered to a pH of about 8.3;

about 0.5% w/v of sodium deoxycholate;

about 0.9% w/v benzyl alcohol; and

about 1% w/v of sodium chloride.

Para. BM. The method of Para. AZ, wherein the DCA is administered as a precipitation stable aqueous composition consisting essentially of:

an aqueous solution buffered to a pH of about 8.3;

about 1% w/v of sodium deoxycholate;

about 0.9% w/v benzyl alcohol; and

about 1% w/v of sodium chloride.

Para. BN. The method of any one of Paras. AZ-BM, wherein the DCA is administered by injection.

Para. BO. The method of any one of Paras. AZ-BN, wherein the DCA is administered by a plurality of injections.

What is claimed is:

1. A method of treating accumulated fat in a patient in need thereof, comprising administering into the accumulated fat a therapeutically effective amount of deoxycholic acid (DCA) or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea.

2. The method of claim 1, wherein the DCA is administered as a precipitation stable aqueous composition consisting essentially of from about 0.4% w/v to less than about 2% w/v of a salt of deoxycholic acid, wherein said composition is maintained at a pH of about 8.1 to about 8.5.

3. The method of claim 1, wherein the DCA is present in an amount from about 0.5% w/v to about 1% w/v.

4. The method of claim 1, wherein the DCA is present in an amount of about 0.5% w/v.

5. The method of claim 1, wherein the DCA is present in an amount of about 1% w/v.

6. The method of claim 1, wherein the excipient is a solvent, a buffer, a preservative, a lyophilization aid, or any combination thereof.

7. The method of claim 1, wherein the excipient is a solvent.

8. The method of claim 1, wherein the excipient is a preservative.

9. The method of claim 1, wherein said excipient is 0.9% benzyl alcohol.

10. The method of claim 1, wherein said composition has a pH of about 8.3.

11. The method of claim 1, wherein said salt is an alkali metal salt.

12. The method of claim 11, wherein said alkali metal salt is sodium.

13. The method of claim 1, wherein the DCA is administered as a precipitation stable aqueous composition consisting essentially of:
a sterile aqueous solution buffered to a pH of about 8.3;
about 0.5% w/v of sodium deoxycholate;
about 0.9% w/v benzyl alcohol; and
about 1% w/v of sodium chloride.

14. The method of claim 1, wherein the DCA is administered as a precipitation stable aqueous composition consisting essentially of:
an aqueous solution buffered to a pH of about 8.3;
about 1% w/v of sodium deoxycholate;
about 0.9% w/v benzyl alcohol; and
about 1% w/v of sodium chloride.

15. The method of claim 1, wherein the DCA is administered by injection.

16. The method of claim 1, wherein the DCA is administered by a plurality of injections.

17. A method of reducing accumulated fat in a patient in need thereof, comprising administering into the accumulated fat a therapeutically effective amount of deoxycholic acid (DCA) or a pharmaceutically acceptable salt thereof and at least one pharmaceutically acceptable excipient, wherein the accumulated fat results from or causes one or more of excess axillary fat, lateral periaxillary fat, pre axillary fat, post axillary fat, anterior periaxillary fat, posterior periaxillary fat, fat on the upper back, bra fat, back of arm fat, fat on the anterolateral flank, love handle, medial knee fat, inner upper thigh fat, outer upper thigh fat, calf fat, fat around the ankles, excess fat on the face, including one or more of intraorbital fat, periorbital fat, malar fat and/or jaw fat, stomach fat (including, but not limited to periumbilical fat, fat above periumbilical area, fat below periumbilical area, or any combination of two or more thereof), excess fat on the buttocks, mons pubis fat, excess fat around the ankles, fat on the upper back of the thigh, excess fat on the foot, pseudogynocomastia fat, lipoma, lipodystrophy (such as Dunning-type lipodystrophy), lipomatosis such as familial multiple lipomatosis, post-liposuction fat deposits, and obstructive sleep apnea.

18. The method of claim 17, wherein the DCA is administered as a precipitation stable aqueous composition consisting essentially of from about 0.4% w/v to less than about 2% w/v of a salt of deoxycholic acid, wherein said composition is maintained at a pH of about 8.1 to about 8.5.

19. The method of claim 17, wherein the DCA is present in an amount from about 0.5% w/v to about 1% w/v.

20. The method of claim 17, wherein the DCA is present in an amount of about 0.5% w/v.

21. The method of claim 17, wherein the DCA is present in an amount of about 1% w/v.

22. The method of claim 17, wherein the excipient is a solvent, a buffer, a preservative, a lyophilization aid, or any combination thereof.

23. The method of claim 17, wherein the excipient is a solvent.

24. The method of claim 17, wherein the excipient is a preservative.

25. The method of claim 17, wherein said excipient is 0.9% benzyl alcohol.

26. The method of claim 17, wherein said composition has a pH of about 8.3.

27. The method of claim 17, wherein said salt is an alkali metal salt.

28. The method of claim 27, wherein said alkali metal salt is sodium.

29. The method of claim 17, wherein the DCA is administered as a precipitation stable aqueous composition consisting essentially of:
a sterile aqueous solution buffered to a pH of about 8.3;
about 0.5% w/v of sodium deoxycholate;
about 0.9% w/v benzyl alcohol; and
about 1% w/v of sodium chloride.

30. The method of claim 17, wherein the DCA is administered as a precipitation stable aqueous composition consisting essentially of:
an aqueous solution buffered to a pH of about 8.3;
about 1% w/v of sodium deoxycholate;
about 0.9% w/v benzyl alcohol; and
about 1% w/v of sodium chloride.

31. The method of claim 17, wherein the DCA is administered by injection.

32. The method of claim 17, wherein the DCA is administered by a plurality of injections.
